(19) United States
(12) Patent Application Publication
(10) Pub. No.: US 2025/0110081 A1
(43) Pub. Date: Apr. 3, 2025

(54) MEASURING SYSTEM FOR A MONITORING SYSTEM

(71) Applicant: Dräger Safety AG & Co. KGaA, Lübeck (DE)

(72) Inventors: Tilman von BLUMENTHAL, Lübeck (DE); Hans-Ullrich HANSMANN, Lübeck (DE); Henning GERDER, Lübeck (DE); Robert JAHNS, Lübeck (DE); Christoph OSTERLOH, Lübeck (DE)

(21) Appl. No.: 18/895,701

(22) Filed: Sep. 25, 2024

(30) Foreign Application Priority Data
Sep. 28, 2023 (DE) 10 2023 126 406.0

(51) Int. Cl. G01N 27/74; G01N 33/00

(52) U.S. Cl. CPC G01N 27/74; G01N 33/0016

(57) ABSTRACT

A measuring system (60, 69, 68) determines gas concentrations as part of a monitoring system for breathing gases or a breathing gas mixture (10). The measuring system (60, 69, 68) is capable of determining an oxygen concentration (908) in the breathing gas mixture (10) under variable pressure conditions (599).

### CROSS REFERENCE TO RELATED APPLICATIONS

This application claims the benefit of priority under 35 U.S.C. § 119 of German Application 10 2023 126 406.0, filed Sep. 28, 2023, the entire contents of which are incorporated herein by reference.

### TECHNICAL FIELD

The present invention relates to a paramagnetic measuring system for determining gas concentrations for a monitoring system. In particular, the invention relates to a measuring system which is used as part of a monitoring system for monitoring the breathing gas supply with a sufficient concentration of oxygen of a person in an aircraft or an underwater vehicle or a diver. According to the invention, the measuring system is able to detect an oxygen concentration in the breathing gas mixture during an operation (mission). To determine the oxygen concentration in gases, paramagnetic methods are often used, which are based on the fact that oxygen molecules are paramagnetic due to their permanent magnetic dipole moment, whereas most other gases have diamagnetic properties.

### BACKGROUND

It is generally known that the thermal conductivity of paramagnetic gases changes under the influence of magnetic fields. The cause of this behavior is apparently the fact that paramagnetic gases have a permanent magnetic moment, which, however, does not normally appear externally (it is not usually observed) due to the thermal molecular motion of the gas molecules.

However, a sufficiently strong external magnetic field ensures that the magnetic dipole moments of the individual molecules are aligned. On the one hand, this causes a change in susceptibility, which results in an increase in the magnetic flux; on the other hand, a certain molecular arrangement is established in the gas, which limits the degrees of freedom and thus the possibilities of transferring thermal energy to neighboring molecules via collisions.

As a result, the thermal conductivity of the gas changes to a small extent.

A basic principle for measuring oxygen in a measuring chamber using heat conduction changes in connection with paramagnetism is described in U.S. Pat. No. 6,430,987B1.

US20230114548A1 describes a way of determining the presence of an anesthetic gas in the breathing gas mixture in addition to measuring oxygen.

US 2023 011 4548 A1 shows a measuring system for determining gas concentrations in a gas mixture of a gas sample by utilizing thermal conductivities and paramagnetic effects of thermal conductivities in the gas mixture. A circuit arrangement supplies measured values with an AC signal component and with a DC signal component to a computing and control unit. Using data sets, an oxygen concentration in the gas mixture is determined by means of normalization of AC voltage signal components and a concentration of another gas in the gas mixture is determined by means of normalization (standardization) of DC voltage signal components.

A monitoring system for monitoring pilots and/or co-pilots is known from US20210405008A1 (US20210405008A1 is incorporated herein by reference).

The monitoring system is preferably configured as part of the equipment as a self-sufficient and mobile, body-worn unit with its own power supply.

Typical applications of paramagnetic measuring systems are known from the field of medical technology for analyzing oxygen concentrations in breathing gas mixtures. These typical applications of the measuring systems take place in a typical ambient pressure range of 1000 hPa+/−150 hPa. In this pressure range, the effects caused by the pressure on the thermal conductivity in a gas mixture do not yet have a major impact on the measurement accuracy of the oxygen concentration. With larger deviations from the normal pressure of 1013 mBar (corresponding to 1013 hPa) under normal conditions (sea level, NN (Normal-Null)), the different molecular sizes of the various gas components present in the gas mixture can have pressure-dependent effects.

One pressure-dependent effect is the collision probability of molecules as a function of the pressure-dependent total number of molecules per spatial unit.

Jet aircraft are used and flown at altitudes of up to 15,000 feet or more above the ground, i.e. at altitudes above 4,500 meters. Depending on the time of year, weather conditions and latitude, the prevailing pressure conditions there are in the region of 575 hPa. Due to the rapid flight maneuvers of jet aircraft, pressure levels can also change rapidly within a very short time. A descent from above 4,000 meters to below 1,000 meters, for example, results in a pressure increase with a pressure gradient of approximately +300 hPa, which can affect the functions of the measuring system. Therefore, there is a need for a measuring system, which is able to detect a concentration of oxygen in a gas mixture during aircraft flight operations with low accuracy restrictions under changing pressure conditions, even at higher altitudes.

### SUMMARY

It is an object of the present invention to provide and specify a measuring system that is improved for pressure ranges below normal conditions, for example for an application of the monitoring system in monitoring the breathing gas supply in aircraft.

A further task is to provide and specify an improved measuring system for pressure ranges far above normal conditions, for example for an application of the monitoring system in monitoring the breathing gas supply in underwater vehicles or diving applications.

These and other objects are attained, and problems are solved by a measuring system with features according to the invention.

Further features and details of the invention are shown and described in the description and the drawings and in the claims.

A measuring system according to the invention has at least the following components for solving the tasks:
a module for gas transportation,
a sample gas line (sample line/measurement gas line),
a measuring device
   with a measuring element in a measuring chamber,
   with a pressure sensor,
   with a magnet arrangement with an electromagnet and a coil,
a calculation and control unit with an assigned data memory, and
a circuit arrangement.

The gas transport module is configured to draw in defined quantities or partial quantities (samples) of breathing gas mixture from a measuring location, in particular from a person's breathing mask and/or from the cabin or cockpit, by means of the sample gas line and to transport it to the monitoring system or into the measuring chamber. For this purpose, the gas transport module is pneumatically and/or fluidically connected to the measuring location and the measuring chamber by means of the sample gas line. The measuring element is configured as a planar semiconductor or silicon element (MEMS, semiconductor chip) with a membrane and has a heating structure on the membrane and a heat conduction measuring unit at a measuring point.

The heat conduction measuring unit is configured as a thermocouple or as a thermopile. The heating structure can, for example, be configured as a resistive heating element.

The calculation and control unit is configured to detect thermoelectric voltage signals from the measuring device provided by the measuring element.

The measuring device has a pressure sensor which is arranged in or on the measuring chamber or the measuring element in such a way as to detect a measured pressure value which indicates a pressure level inside the measuring chamber or a pressure level of a breathing gas mixture flowing into the interior of the measuring chamber. The measuring device is configured with the pressure sensor to determine a measured pressure value which indicates a pressure level inside the measuring chamber.

Together with the circuit arrangement, the measuring device is configured to heat the heating structure on the membrane of the measuring element. The measuring element is arranged in the measuring chamber in such a way that a quantity of the breathing gas mixture can be supplied to the measuring element as a gas sample. The measuring element is heated to a working temperature that is higher than the temperature of the quantity of breathing gas mixture in the measuring chamber. The measuring device is configured with the electromagnet, the coil and the circuit arrangement to generate a magnetic field acting on the measuring element. If a paramagnetic gas is present in the breathing gas mixture at the measuring element, the heat conduction changes under the influence of the magnetic field in proportion to the proportion of paramagnetic gas. If, for example, oxygen is present in the breathing gas mixture, the temperature rises, resulting in an increase in the thermoelectric voltage signals that can be detected at the heated measuring element. The calculation and control unit and the circuit arrangement can be used to monitor, control and regulate the temperature control of the measuring element with this put into practice:
as operation with a constant heating voltage;
as operation with a constant heating current; and
as operation with a constant heat output.

The circuit arrangement is configured for signal processing of the thermoelectric voltage signals with provision of measured values with an AC voltage signal component and with a DC voltage signal component to the calculation and control unit.

The signal processing is configured using suitable circuits, such as lock-in systems, high-pass/low-pass filter circuits for splitting the thermoelectric voltage signals into AC voltage signal components and DC voltage signal components. Suitable variants for the operation of the measuring elements and the signal processing of the thermoelectric voltage signals can be found, for example, in US 2023 0114 548 A1 (US 2023 0114 548 A1 is incorporated herein by reference). The circuit arrangement is configured to provide measured values of the measuring element with an AC voltage signal component U_{X˜} and with a DC voltage signal component U_{X=} to the calculation and control unit. The calculation and control unit is configured with the circuit arrangement to generate a modulated magnetic field acting on the measuring element by activating and deactivating the electromagnet with the coil arrangement.

The calculation and control unit is configured with the circuit arrangement for splitting the thermoelectric voltage signals into AC voltage signal components U_{X˜} and DC voltage signal components U_{X=}. The AC voltage signal components represent heat conduction components that are modulated and influenced by the magnetic field. Such an influence is given by the effect of paramagnetism in the heat conduction for the proportions of oxygen in the breathing gas mixture. The DC voltage signal components represent portions of the heat conduction in the breathing gas mixture that cannot be influenced by the magnetic field.

The calculation and control unit is configured to determine the current oxygen concentration in the breathing gas mixture from the ratio of the modulated heat conduction components to the heat conduction components that are not modulated by the magnetic field. In the ideal case, the ratio of the modulated heat conduction components to the heat conduction components that are not modulated by the magnetic field represents the current oxygen concentration in cases in which a binary gas mixture is given, i.e. for example a gas mixture consisting of a relevant proportion of oxygen and a predominant proportion of nitrogen with negligible proportions of carbon dioxide, noble gases and with a low water content, for example dry ambient air, technical or medical compressed air. In reality, however, a breathing gas mixture is not a binary gas mixture, as the concentrations of water vapor and carbon dioxide in particular are significantly higher during exhalation than during inhalation.

To take into account gas mixtures, such as breathing gas mixtures with more than two relevant gas components, characteristic curves are required to correct the ratio of the modulated heat conduction components to the heat conduction components that are not modulated by the magnetic field. The number and selection of the required characteristic curves is crucial, but also means increasing complexity in systemic or individual calibration and in measurement operation. Coefficients for linear or higher derivatives of the influences are preferably calculated from the characteristic curves.

For concentrations of oxygen (O₂) in a breathing gas mixture, the following approach for setting up characteristic curves according to the following formula 1 is used as an example:

$${C}_{O 2} = {K}_{1} * {a}_{1} / {b}_{1} + {K}_{2} * {( {a}_{1} / {b}_{1} )}^{2} + {K}_{3} * {a}_{1} + {K}_{4} * {a}_{1}^{2} + {K}_{5} * {b}_{1} + {K}_{6} * {b}_{1}^{2} + {K}_{7} * {c}_{1} + … ,$$

where K_{X} represents the coefficients of the components present in the breathing gas and the variables a₁, b₁, c₁, . . . each represent measured variables of heat conduction, such as the heat conduction dependent on the magnetic field and the heat conduction of the gases in the gas mixture independent of the magnetic field. In addition, the variables a₁, b₁, c₁, . . . can also include information on vapor pressures, dew points of the gases in the gas mixture or their water content in the gas mixture. These coefficients are available to the measuring system for the subsequent measuring operation.

According to the invention, the calculation and control unit is configured to include an effect of the ambient pressure in the determination of the current oxygen concentration in the breathing gas mixture.

In the design of the measuring system, characteristic curves are recorded which reflect, for example, the influence of material properties of the measuring elements, effects and boundary conditions of inflow, flow and/or outflow of supplied quantities of breathing gas mixture into the measuring chamber, of material properties and constructive structures of the measuring chamber or by processes of semiconductor production of the measuring elements (MEMS), for example in the form of possible scattering in the membrane thickness of the measuring elements.

In order to equalize or compensate for these influences, the thermovoltage signals with AC signal components and DC signal components and/or the ratios of AC signal components to DC signal components for typical breathing gas mixtures with oxygen, carbon dioxide, nitrogen, moisture or water vapor can be determined for different compositions of the breathing gas mixture, water vapor, the thermovoltage signals with AC signal components and DC signal components and/or the ratios of AC signal components to DC signal components for typical breathing gas mixtures with oxygen components in the breathing gas mixture can be measured as part of a functional test and stored and saved in a data memory (RAM, ROM) as data sets, which then characterize this measuring system individually or systemically in the form of a characteristic curve, a set of characteristic curves or as a characteristic diagram (characteristic field).

The characteristic curves or the characteristic diagram are usually recorded under normal conditions of pressure and temperature or under typical defined ambient conditions of pressure and temperature, for example according to the approach described in Formula 1.

When the measuring system is used to determine gas concentrations when supplying people with the breathing gas mixture, the data sets from the data memory (data storage device) are then used to determine the current oxygen concentration in the breathing gas mixture with the highest possible accuracy from the AC signal components and DC signal components. If, as intended by the task of the present invention, the measuring system is also to be used at higher altitudes with lower pressure levels compared to normal conditions and also under rapidly changing pressure situations during flight operations, it is necessary to form the characteristic curves, characteristic diagrams and characteristic curve groups (characteristic curve arrays) not only at one typical pressure level in order to maintain the measurement accuracy of the oxygen concentration, but at at least two pressure levels in order to take into account the extended pressure range in the measuring system, as can occur during flight operations, and to include the pressure-dependent effect on the thermal conductivity of different gas proportions in the breathing gas mixture, even for pressure conditions deviating from sea level and the earth's surface, in the signal evaluation and determination of a current oxygen concentration in the breathing gas mixture. Operation with fluctuations in the pressure level inside the measuring chamber caused by flight operations and flight maneuvers should also be possible.

In measuring mode, the pressure sensor plays a key role in continuously determining the current pressure level inside the measuring chamber or the pressure level of the breathing gas mixture—as well as fluctuations in the pressure level—and making it available to the calculation and control unit.

In accordance with the invention, the pressure-dependent effect can be included in the determination of the oxygen concentration in the manner described below. According to the invention, the thermoelectric voltage signals are recorded to form characteristic curves or diagrams of the measuring elements at at least two defined and different pressure levels. This can take place, for example, as part of a test of the measuring system following assembly or configuration or also as part of a subsequent adjustment or calibration. The thermoelectric voltage signals with AC voltage and DC voltage signal components and/or the ratio of AC voltage to DC voltage signal component as a quotient are determined for different and typical compositions of moisture, oxygen, nitrogen, carbon dioxide in the breathing gas mixture as a first situation in the breathing gas mixture at a first pressure level and stored as a characteristic curve, a set of characteristic curves or a characteristic diagram as a first data set in the data memory. The thermovoltage signals with AC and DC voltage signal components and/or the ratio of AC voltage to DC voltage signal component as a quotient are determined for different and typical compositions of moisture, oxygen, nitrogen, carbon dioxide in the breathing gas mixture as a second situation in the breathing gas mixture at at least one further second pressure level and stored as a characteristic curve, a set of characteristic curves or a characteristic diagram as a second data set in the data memory.

The calculation and control unit is configured on the basis of the AC and DC voltage signal components,
taking into account (based on/including) the measured pressure value, which indicates the current pressure level inside the measuring chamber,
taking into account (based on/including) the first data set and
taking into account (based on/including) the second data set to carry out a determination of a current oxygen concentration in the breathing gas mixture.

The calculation and control unit is thus configured to determine an output signal which indicates an oxygen concentration in the breathing gas mixture on the basis of the AC and DC voltage signal components, the current pressure level inside the measuring chamber and the first and second data sets. The calculation and control unit can, for example, apply corresponding characteristic curves for the at least two pressure levels according to the approach described above in Formula 1. The coefficients for at least two pressure levels can then be formed accordingly as coefficients Pₓ, Qₓ and the measured variables a₁, b₁, c₁ of the heat conduction for the at least two further pressure levels can be formed as a₂, b₂, c₂, or a₃, b₃, c₃ and stored accordingly in the first or second data set and kept ready for measurement operation.

The first pressure level is taken into account as the basis for the first data set, for example, using the approach shown in Formula 2 below:

$${C}_{O 2} = {P}_{1} * {a}_{2} / {b}_{2} + {P}_{2} * {( {a}_{2} / {b}_{2} )}^{2} + {P}_{3} * {a}_{2} + {P}_{4} * {a}_{2}^{2} + {P}_{5} * {P}_{1} + {P}_{6} * {b}_{2}^{2} + {P}_{7} * {c}_{2} + … .$$

The additional or second pressure level is taken into account as the basis for the second data set, for example, using the approach shown in Formula 3 below:

$${C}_{O 2} = {Q}_{1} * {a}_{3} / {b}_{3} + {Q}_{2} * {( {a}_{3} / {b}_{3} )}^{2} + {Q}_{3} * {a}_{3} + {Q}_{4} * {a}_{3}^{2} + {Q}_{5} * {Q}_{1} + {Q}_{6} * {b}_{3}^{2} + {Q}_{7} * {c}_{3} + … .$$

The calculation and control unit is configured to provide the output signal that indicates the specific oxygen concentration.

The following is a summarized description of the inventive embodiment of the measuring system for determining gas concentrations when supplying people with breathing gas mixtures. The measuring system has a module for gas transport, a sample gas line and a measuring device. The measuring device has a measuring element in a measuring chamber.

The gas transport module is configured to supply a quantity of a breathing gas mixture to the measuring element in the measuring chamber. The measuring device has a calculation and control unit with an associated data memory and a circuit arrangement. The measuring device also has a magnet arrangement with an electromagnet and a coil.

The measuring device is configured with the circuit arrangement for operating a heating structure on a membrane of the measuring element in order to transfer defined quantities of heat into the breathing gas mixture or to partial quantities of the breathing gas mixture in the measuring chamber.

The measuring device is configured with the pressure sensor to determine a measured pressure value, which indicates a pressure level inside the measuring chamber. The measuring device is configured with the electromagnet, the coil and the circuit arrangement to generate a magnetic field acting on the measuring element. The circuit arrangement is configured to provide measured values of the measuring element with an alternating voltage signal component and with a direct voltage signal component to the calculation and control unit.

A first data set with first values and at least one second data set with further values are stored in the data memory.

The first data set indicates a situation of gases or breathing gas mixtures at a first pressure level.

The second data set indicates a situation of gases or breathing gas mixtures at a second pressure level.

In particular embodiments, at least one further or third data set with further values for a third or further pressure levels can be stored in the data memory, which indicate a third or further situation in the breathing gas mixture at a third pressure level or a further pressure level.

In order to improve the measurement operation for low pressure ranges below normal conditions, as they can occur in aircraft (jet aircraft, helicopters) or in flight applications (parachutists), as well as for large pressure ranges above normal conditions, as they can occur in underwater vehicles (submarines) or in diving applications (mine divers, maintenance divers, welding divers), the data memory can also contain data sets for the low or large pressure ranges common in these applications.

In the further course of the description of “low pressure levels in flight operations”, a comparable technical implementation can also be read for “high pressure levels in diving operation”. In both applications (flight operation, diving operation), the calculation and control unit is configured and capable of using the data sets from the data memory to determine the oxygen concentration with compensation for pressure influences.

The calculation and control unit is configured to determine a current oxygen concentration in the breathing gas mixture on the basis of the alternating voltage signal components, direct voltage signal components, taking into account the measured pressure value, which indicates a current pressure level inside the measuring chamber, taking into account the first data set and the second data set.

The calculation and control unit is configured to provide an output signal that indicates the current oxygen concentration in the breathing gas mixture.

Embodiments show how the calculation and control unit can be configured to include the current measured pressure value and the data sets stored in the data memory in the determination of the current oxygen concentration in the breathing gas mixture during measurement operation (measurement mode).

The following examples show variants for taking into account the first and second data set for a current pressure level in the breathing gas supply of 703 hPa during flight operations corresponding to a flight altitude of approximately 3000 meters in the determination of the current oxygen concentration. In this example, the first data set comprises a characteristic curve, a set of characteristic curves or a characteristic diagram with a reference level of the ambient pressure of 650 hPa.

In this example, the second data set comprises a characteristic curve, a set of characteristic curves or a characteristic diagram with a reference level of 1050 hPa.

In a first variant for taking into account the first and second data set, one approach would be to use the second data set in measurement mode as long as the current pressure level in the breathing gas mixture is above 650 hPa and then switch to the first data set as soon as the current pressure level of the breathing gas mixture is below 650 hPa.

In a corresponding preferred embodiment, the calculation and control unit is configured to include (take into account) the current measured pressure value, to include (take into account) the first data set and to include (take into account) the second data set or the third or even further data sets by means of a switchover between the data sets to determine the current oxygen concentration.

In a second variant according to a further preferred embodiment for taking into account the first and second data set (including the first and second data set in determining the current oxygen concentration), one approach would be to perform an interpolation in measurement mode based on the data of the first data set (assigned to 650 hPa) and the second data set (assigned to 1050 hPa) and to generate an interpolated data set in operation that is representative of the current pressure level of 703 hPa with regard to different and typical compositions of moisture, oxygen, nitrogen, carbon dioxide in the breathing gas mixture.

Preferably, linear interpolation can be used for a number of two data sets.

To explain a third variant in accordance with a further preferred embodiment, it is assumed as an example—as in the two previous examples—that the first data set has a characteristic curve or a characteristic diagram with a pressure level of 1050 hPa as the data basis and the second data set has a characteristic curve or a characteristic diagram with a pressure level of 650 hPa as the data basis. In this third example, the pressure level prevailing in measurement mode is assumed to be even lower than in the previous examples at 703 hPa, for example 600 hPa, which would correspond to a further increase in the altitude of the aircraft to approx. 4200 meters compared to the first and second examples. In this third variant, one approach would be to extrapolate the data from the first data set (assigned to 1050 hPa) and the second data set (assigned to 650 hPa) to the current pressure level of 600 hPa during measurement operation and to generate an extrapolated data set during operation that is representative of the current pressure level of 600 hPa with regard to different and typical compositions of humidity, oxygen, nitrogen and carbon dioxide in the breathing gas mixture.

Preferably, linear extrapolation can be used for a number of two data sets.

In further preferred embodiments, the thermoelectric voltage signals, their AC and DC voltage signal components and/or their quotient for different and typical compositions of oxygen, nitrogen and carbon dioxide in the breathing gas mixture can be determined for further pressure levels and stored as characteristic curves, characteristic curve sets or characteristic diagrams as further data sets in the data memory. This makes it possible, for example, to switch between different characteristic curves with greater precision based on the measured pressure value at the current measurement time in measurement mode. Alternatively, it can also be advantageously achieved, for example, that interpolations and extrapolations can be carried out on the basis of the measured pressure value at the current measurement time in measurement mode using a large number of interpolation points on the characteristic curves with improved approximation to the real pressure-dependent effect.

In addition, non-linear interpolations or extrapolations can also be used for more than three or five interpolation points and form preferred embodiments.

In a preferred embodiment, the interpolation or extrapolation may be performed as a linear interpolation or extrapolation or a sectional linear interpolation or extrapolation by the calculation and control unit. In a preferred embodiment, the interpolation or the extrapolation can be performed as a non-linear interpolation or non-linear extrapolation, in particular as a quadratic or as a cubic interpolation or extrapolation by the calculation and control unit.

In further preferred embodiments, data or knowledge of ratios and time delays of pressure rise or fall between ambient pressure or cabin pressure and the pressure required for the pilot to breathe can be used to determine the pressure drop or rise.

The possible effects of the effective pressure in the breathing mask on the thermoelectric voltage signals can be predicted and predictively included in the determination of the oxygen concentration by the calculation and control unit. Such effects are, for example, changes in the thermal conductivities in the gas mixture. For this purpose, according to this further preferred embodiment, an acceleration sensor (accelerometer) can be arranged in or on the measuring system, which is configured to determine measurement data on accelerations, on a current spatial position of the measuring system or changes in the spatial position and to provide them to the calculation and control unit. The calculation and control unit can be configured to predictively include the current measured pressure value and the first and second data sets in the determination of the current oxygen concentration on the basis of the measurement data from the acceleration sensor. In an alternative embodiment, the acceleration sensor can be arranged in or on the monitoring system and assigned to the measuring system.

With the help of an acceleration sensor—in particular a 3-axis acceleration sensor arranged on the measuring system—flight maneuvers can be identified and possible future expected pressure changes can be predicted or estimated by the calculation and control unit.

In this way, for example, a descent maneuver can be identified and the pressure increase of 300 hPa, which results from a descent by an altitude difference of 3,000 m, for example from above 4,000 meters to below 1,000 meters, can thus be predicted.

This predetermination can enable the calculation and control unit to take into account various flight maneuvers (take-off, landing, descent, looping), the conditions known and/or predictable for the maneuvers and time delays of pressure increase or pressure drop between the ambient pressure or cabin pressure and the pressure in the breathing mask effective for the pilot's breathing as pressure-dependent effects when determining the current oxygen concentration in the breathing gas mixture.

The pressure-dependent effects can thus be taken into account according to the invention in such a way that a change in the pressure level in the breathing gas mixture during a flight maneuver can be included in the determination of the current oxygen concentration, which has a predictable—i.e. predictive-effect on the measuring system and thus on the gas mixture in the measuring chamber towards the end of a flight maneuver (descent, ascent).

In a further preferred embodiment, the calculation and control unit can be configured to include in the determination of the oxygen concentration a measured value of a temperature sensor, which indicates a temperature level in the measuring chamber, and/or information provided with regard to a temperature level in the breathing gas mixture.

In a comparable procedure to the approaches for different temperature levels shown with formulae 1, 2, 3 as a basis for realizing characteristic curves, characteristic diagrams or sets of characteristic curves stored in data sets in the data memory, further approaches with formulae 4 and 5 can be specified in this preferred embodiment in order to additionally include at least two different temperature levels in interpolations, extrapolations based on the data sets or switching between the data sets when determining the oxygen concentration as data sets.

For example, the coefficients Rₓ, Sₓ can be formed for two different temperature levels and the measured variables a₁, b₁, c₁ of the heat conduction for the two temperature levels can be formed as a₂, b₂, c₂, or a₃, b₃, c₃, stored accordingly in further data sets and kept ready for measurement operation. A temperature level is taken into account as the basis for a third data set, for example, using the approach shown in Formula 4 below:

$${C}_{O 2} = {R}_{1} * {a}_{2} / {b}_{2} + {R}_{2} * {( {a}_{2} / {b}_{2} )}^{2} + {R}_{3} * {a}_{2} + {R}_{4} * {a}_{2}^{2} + {R}_{5} * {R}_{1} + {R}_{6} * {b}_{2}^{2} + {R}_{7} * {c}_{2} + … .$$

Another temperature level can be taken into account as the basis for a fifth data set, for example, using the approach shown in Formula 5 below:

$${C}_{O 2} = {S}_{1} * {a}_{3} / {b}_{3} + {S}_{2} * {( {a}_{3} / {b}_{3} )}^{2} + {S}_{3} * {a}_{3} + {S}_{4} * {a}_{3}^{2} + {S}_{5} * {S}_{1} + {S}_{6} * {b}_{3}^{2} + {S}_{7} * {c}_{3} + … .$$

In this way, the calculation and control unit is able to take into account the dependence of the reduction in air pressure on the temperature at altitude. The following formula 6 with two examples should serve as an explanation. Formula 6 below describes the general relationship between air pressure, altitude and temperature:

$$p ( {h}_{1} ) = p ( {h}_{0} ) {( 1 - \frac{0 , 0065 \cdot Δ h}{T ( {h}_{0} )} )}^{5 , 255}$$

Based on a reference altitude h₀ (0.00m, sea level, NN) with a given pressure level p(h0) of 1013 mBar (corresponding to 1013 hPa) and an assumed reference temperature T(h0) of 288.15 K (corresponding to 15° C.), a pressure level p(h₁) of approx. 617 hPa results at an altitude h₁ of approx. 4000 meters above sea level. At the same altitude of approx. 4000 meters, a temperature of 5° C. results in a pressure level p(h₁) of approx. 605 hPa.

This enables the calculation and control unit to take into account not only the pressure-dependent effect resulting from the altitude for determining the current oxygen concentration during operation of the measuring system when using the thermoelectric voltage signals, but also the dependence of the pressure-dependent effect on the temperature level prevailing during operation.

In a further preferred embodiment, the calculation and control unit can be configured to include in the determination of the oxygen concentration a measured value of a humidity sensor, which indicates a humidity situation in the breathing gas mixture in the measuring chamber, and/or information provided with regard to a current humidity content in the breathing gas mixture. The current moisture content in the breathing gas mixture can, for example, be included in the data sets based on formulae 1 to 5 or directly or indirectly in formulae 1 to 5, which have the measured variables of heat conduction a₁, a₂, a₃, b₁, b₂, b₃, c₁, c₂, c₃ as parameters or information on vapor pressures, dew points of the gases in the gas mixture or their water content in the gas mixture.

In a further preferred embodiment, a temperature sensor can be arranged in or on the measuring chamber.

In a further preferred embodiment, a humidity sensor can be arranged in or on the measuring chamber.

In a further preferred embodiment, the calculation and control unit can be configured to work together with a measuring chamber heater to control the temperature of the measuring chamber to a temperature level with a predetermined temperature difference above the ambient temperature, for example above the temperature in the cockpit of the aircraft. In this way, condensation in the measuring chamber can be prevented with a low expenditure of heating energy. To implement this further preferred embodiment in practice, it is necessary to provide the calculation and control unit with a temperature value at an interface, which indicates the ambient temperature and/or the cockpit temperature.

In a further preferred embodiment, the calculation and control unit can be configured to initiate temperature control of the measuring chamber to a temperature level significantly above a typical body temperature of the pilot and the current ambient temperature for a predetermined period of time after a predetermined time interval has elapsed.

In this way, condensation in the measuring chamber can be reliably prevented with a small amount of heating energy during the specified period of time. In addition, the influence of the temperature dependencies of the thermal conductivities of the gases oxygen, nitrogen and carbon dioxide involved in the breathing gas mixture as well as the influence of humidity on the determination of the current oxygen concentration in the breathing gas mixture can be reduced for the duration of the specified time period. In this way, a current oxygen concentration value of the breathing gas mixture can be determined from time to time, which is characterized by the fact that it has improved accuracy compared to the oxygen concentration value determined by means of switching, interpolation or extrapolation on the basis of the first and second data set.

In a preferred embodiment, the calculation and control unit can be configured in cooperation with a measuring chamber heating system to perform temperature control of the measuring chamber to a selected temperature level for a predetermined period of time. The calculation and control unit can be configured to select the temperature level, selected for the specified period of time, using the current temperature value and at least one of the data sets stored in the data memory. This makes it possible, in an advantageous and energy-saving manner, to carry out the temperature control of the measuring chamber with a slight difference to the current temperature level, in that the temperature control of the measuring chamber is carried out at a temperature level which was also present when the first data set or the second or further data sets were generated.

For example: The current temperature in the measuring chamber is 33° C., the third data set comprises a characteristic curve, a set of characteristic curves or a characteristic diagram with a reference temperature of 25° C., the fourth data set comprises a characteristic curve, a set of characteristic curves or a characteristic diagram with a reference temperature of 40° C. The first and second data sets comprise characteristic curves based on a first and second pressure level.

If the measuring chamber is now kept at a stable temperature of 40° C., the fourth data set can be used directly, i.e. without the use of interpolations or extrapolations.

The calculation and control unit can then determine a current oxygen concentration in the breathing gas mixture from the thermoelectric voltage signals with AC voltage signal component and DC voltage signal component, taking into account the stable temperature of 40° C. in the measuring chamber created by the temperature control, exclusively using the first, second and fourth data sets. In this way, a current oxygen concentration value of the breathing gas mixture can be determined continuously or only from time to time during measurement operation, which is characterized by the fact that it has improved accuracy compared to the oxygen concentration value determined by means of switching, interpolation or extrapolation, whereby only a small amount of energy is required for the temporary temperature control.

The invention is explained in more detail in the following description with partial reference to the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of a monitoring system showing some features of a measuring system for determining a concentration; and

FIG. 2 is a schematic view showing further details of the measuring system for determining a concentration.

### DESCRIPTION OF PREFERRED EMBODIMENTS

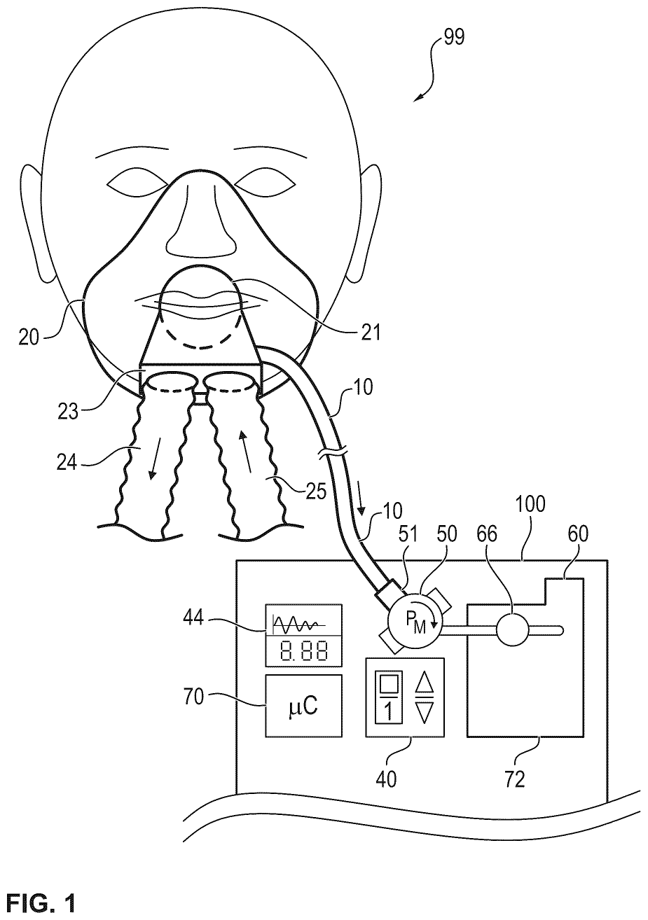

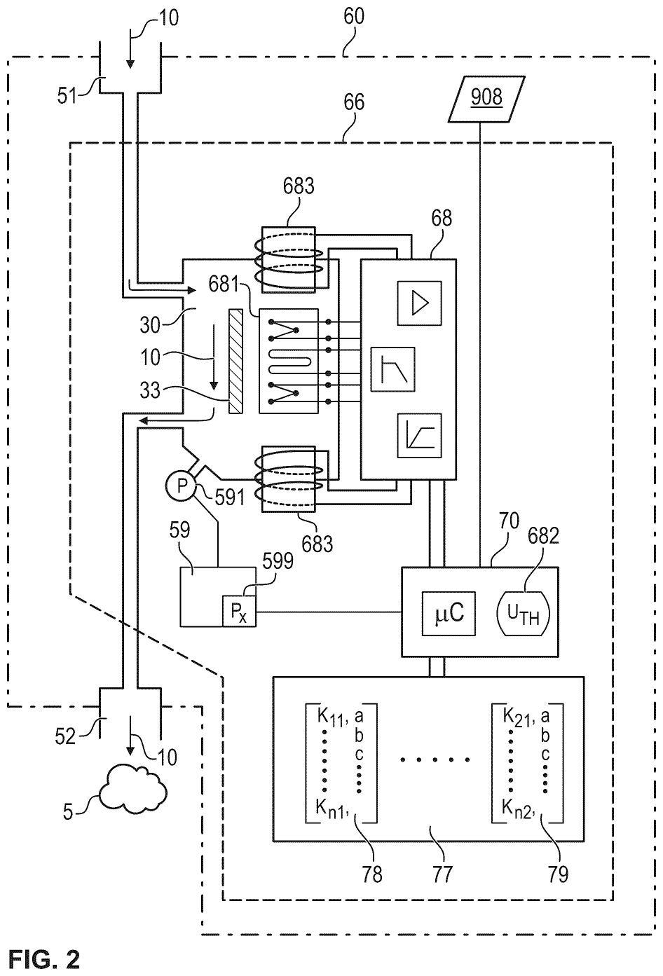

Referring to the drawings, identical elements in FIGS. 1 and 2 are designated with identical reference numerals in FIGS. 1, and 2. FIG. 1 shows a monitoring system 100, showing some features of the measuring system 60 for determining a concentration according to the invention. The monitoring system 100 is connected to a person 99 with a sample gas line 10 by means of a breathing mask 20. The person 99 is an airplane pilot (pilot, co-pilot), boat driver or diver. The breathing mask 20 has a gas connection 21, a connection element 23 and hose lines 24, 25. The hose lines 24, 25 are used for the removal and supply of breathing gases to the person 99. The monitoring system 100 has operating elements 40, display elements 44, a module for gas delivery 50, a measuring system 60 with a measuring device 66. The measuring device 66 has a sensor system, in particular a sensor system for gas analysis, but can also have other sensor systems, for example a sensor system which is configured to measure temperatures, pressures or humidity. The gas delivery module 50 is preferably configured as a pump P_{M}.

In addition, the monitoring system 100 has a control and evaluation unit 70. The operating elements 40, the display elements 44, the sensors and the gas delivery module 50 are connected to the control and evaluation unit 70 via signal and data lines or control lines, which are not shown in FIG. 1. These control lines or signal and data lines can, for example, be configured as a bus system (CAN) or network.

The control and evaluation unit 70 is configured and intended to control and/or actuate the gas delivery module 50 in such a way that breathing gases are delivered from the breathing mask 20 through the sample gas line 10 and a gas inlet 51 to the measuring system 60. Thus, a quantity or partial quantity (a sample) of breathing gas 10 is then available to the measuring device 66 in order to analyze it metrologically and provide it to the control and evaluation unit 70 as measured values.

The control and evaluation unit 70 enables the measured values to be evaluated, processed and displayed on display elements 44.

In optional embodiments, the monitoring system 100 can have further measuring arrangements for temperature and/or pressure measurements in the breathing mask 20 or in the cockpit. In optional embodiments, the monitoring system 100 can have further sensors, for example an inertial measurement system, a gyrometer, an electronic compass or a 2-axis or 3-axis acceleration sensor.

FIG. 2 shows the measuring system 60 with the measuring device 66 and the control and evaluation unit 70 according to FIG. 1 in detail, as well as with an additional data memory 77, which is provided and configured for storing a first data set 78 and at least one further data set 79 and making them available to the control and evaluation unit 70.

Quantities of breathing gas mixture 10 can be conveyed through the module 50 (FIG. 1) for gas transport via a gas inlet 51 to and into a measuring chamber 30 and out again via a gas outlet 52—for example into an environment 5. The control and evaluation unit 70 is provided and configured to carry out a sequence for determining a concentration in the breathing gas mixture 10.

The measuring system 60 has a measuring device 66 with a circuit arrangement 68, which is configured to determine a concentration of oxygen (O₂) in the breathing gas mixture 10 on the basis of paramagnetic effects of thermal conductivity.

The circuit arrangement 68 has a magnet arrangement 683 with an electromagnet and a coil as well as electronic components for operating the coil, magnetic field and for carrying out signal processing and signal filtering. The measuring device 66 has a measuring element 681 with heating elements, thermocouples or thermopiles, which are arranged on a membrane. By means of an interaction of the control and evaluation device 70, circuit arrangement 68 and a magnet arrangement 683, a cyclical effect of a magnetic field on the measuring element 681 is generated during operation.

In addition to the elements schematically indicated in this FIG. 2 for guiding (controlling) a magnetic field, the magnet arrangement 683 has an electromagnet with coils in a coil arrangement, which—like the magnetic field itself—are only indicated in this FIG. 2 for reasons of clarity.

Thermoelectric voltage signals 682 are determined at the thermocouples or thermopiles with and without the influence of the magnetic field as signals with a DC voltage component U_{TH=} and with an AC voltage component U_{TH˜}. The thermoelectric voltage signals 682 U_{TH=}, U_{TH˜} determined at the thermocouples or thermopiles indicate the thermal conductivity of the breathing gas mixture 10 when it comes into contact with the measuring element 681.

The thermoelectric voltage signals 682 U_{TH=}, U_{TH˜} are provided to the control and evaluation unit 70.

The measuring device 66 has a pressure measuring arrangement 59 with a pressure sensor 591 arranged on the measuring chamber 30 for determining a pressure level in the breathing gas mixture 10 as well as its changes in the breathing gas supply to the person 99 (FIG. 1). The control and evaluation unit 70 is configured to continuously record signals from the pressure sensor 591 as a current measured pressure value Pₓ 599, which represents a current pressure level in the breathing gas mixture 10.

An optional measuring chamber heater 33 is arranged on the measuring chamber 30, which is configured to cause at least temporary heating of the measuring chamber 30 in cooperation with the calculation and control unit 70 in order to reduce condensation of moisture in the measuring chamber 30 at least temporarily.

The first data set 78 contains a characteristic curve for various typical proportions of moisture and the gases nitrogen, oxygen and carbon dioxide in the breathing gas mixture 10 with reference to a first pressure level.

The second data set 79 contains a characteristic curve for typical proportions of moisture and the gases nitrogen, oxygen and carbon dioxide in the breathing gas mixture 10 with reference to a further pressure level.

The control and evaluation unit 70 is configured to evaluate the thermoelectric voltage signals 682 U_{TH=}, U_{TH} On the basis of the data of the first data set 78 and the data of the further data set 79, taking into account the current pressure level Pₓ 599, i.e. to form a ratio of the DC voltage component U_{TH=} and the AC voltage component U_{TH˜} in order to determine a current concentration value of oxygen (O₂) in the breathing gas mixture 10 and to provide it as an output signal 908, which indicates the current concentration value of oxygen in the breathing gas mixture 10, and to provide it to an output unit 44 (FIG. 1) or to an optional interface.

In optional embodiments, the measuring system 60 may have further measuring arrangements for temperature and/or pressure measurements and/or humidity measurements.

While specific embodiments of the invention have been shown and described in detail to illustrate the application of the principles of the invention, it will be understood that the invention may be embodied otherwise without departing from such principles.

### REFERENCE LIST

5 Surroundings (environment)
10 Sample gas line (sample line/measurement gas line) for breathing gas mixture
20 Breathing mask
21 Gas connection to breathing mask
23 Connecting element
24, 25 Hose lines
30 Measuring chamber
33 Heating of the measuring chamber
40 Input elements
44 Display elements
50 Module for gas extraction, pump P_{M}
51 Gas inlet
52 Gas outlet
59 Pressure measuring arrangement
591 Pressure sensor
599 Measured pressure value
60 Measuring system
66 Measuring device, sensors
68 Circuit arrangement
681 Measuring element with heating and thermocouple arrangement
682 Thermoelectric voltage signals U_{TH}
683 Magnet arrangement
70 Control and evaluation unit
77 Data memory
78, 79 Data set, data sets
99 Person, pilot, parachutist, boatman, diver
100 Monitoring system
908 Output signal

What is claimed is:

1. A measuring system for determining gas concentrations in a breathing gas mixture for a monitoring system for monitoring a breathing gas supply of an aircraft or an underwater vehicle, the measuring system comprising:
a module for gas transport
a sample gas line;
a measuring device with a measuring element connected to a measuring chamber and comprising a pressure sensor;
a magnet arrangement comprising an electromagnet and a coil;
a calculation and control unit with an associated data memory; and
a circuit arrangement,
wherein the gas transport module is configured to supply a quantity of a breathing gas mixture from the sample gas line to the measuring element in the measuring chamber,
wherein the measuring device with the circuit arrangement is configured to operate a heating structure on a membrane of the measuring element in order to transfer defined quantities of heat into the breathing gas mixture or to partial quantities of the breathing gas mixture in the measuring chamber,
wherein the measuring device with the pressure sensor is configured to determine a measured pressure value which indicates a pressure inside the measuring chamber,
wherein the measuring device with the electromagnet, the coil and the circuit arrangement is configured to generate a magnetic field acting on the measuring element,
wherein the circuit arrangement is configured to provide measured values of the measuring element with an AC voltage signal component and with a DC voltage signal component to the calculation and control unit,
wherein a first data set with first data and at least one second data set with second data are stored in the data memory,
wherein the first data set indicates a first situation of gases in the breathing gas mixture at a first pressure level,
wherein the second data set indicates a second situation of gases or the breathing gas mixture at a second pressure level,
wherein the calculation and control unit is configured to determine a current oxygen concentration in the breathing gas mixture, based on the alternating voltage signal components and direct voltage signal components and based on the measured pressure value, which indicates a current pressure level inside the measuring chamber, the first data set and the second data set,
wherein the calculation and control unit is configured to provide an output signal which indicates the current oxygen concentration in the breathing gas mixture.

2. A measuring system according to claim 1, wherein at least one further or third data set with further values for a third pressure level or further pressure levels is stored in the data memory, which third data set or further data set indicates a third or further situation in the breathing gas mixture at a further pressure level.

3. A measuring system according to claim 1, wherein the calculation and control unit is configured to include the current measured pressure value, the first data set and the second data set, based on a switchover between the data sets, to determine the current oxygen concentration.

4. A measuring system according to claim 2, wherein the calculation and control unit is configured to include the current measured pressure value, the first data set, the second data set, the third and/or further data sets by means of a switchover between the data sets, to determine the current oxygen concentration.

5. A measuring system according to claim 1, wherein the calculation and control unit is configured to include the first data set and the second data set by means of an interpolation based on the values of the data sets for the current measured pressure value, for determining the current oxygen concentration.

6. A measuring system according to claim 2, wherein the calculation and control unit is configured to include the first data set, the second data set, the third and/or further data sets by means of an interpolation based on the values of the data sets for the current measured pressure value for determining the current oxygen concentration.

7. A measuring system according to claim 1, wherein the calculation and control unit is configured to include the first data set and the second data set by an extrapolation based on values of the data sets for the current measured pressure value, for determining the current oxygen concentration.

8. A measuring system according to claim 2, wherein the calculation and control unit is configured to include the first data set, the second data set, the third data set and/or further data sets by an extrapolation based on values of the data sets for the current measured pressure value, for determining the current oxygen concentration.

9. A measuring system according to claim 5, wherein the calculation and control unit is configured to perform the interpolation as a linear interpolation or a sectional linear interpolation, or is configured to perform the interpolation as a non-linear interpolation, as a quadratic interpolation or as a cubic interpolation.

10. A measuring system according to claim 7, wherein the calculation and control unit is configured to perform the extrapolation as a linear extrapolation or a sectional linear extrapolation, or is configured to perform the extrapolation as a non-linear extrapolation, as a quadratic extrapolation or as a cubic extrapolation.

11. A measuring system according to claim 1, further comprising an acceleration sensor, which is arranged in or on the measuring system and which is configured to determine measurement data on accelerations, on a current spatial position of the measuring system or changes in the spatial position and to provide determined measurement data to the calculation and control unit, wherein the calculation and control unit is configured to predictively include a current measured pressure value, the first data set and second data set in the determination of the current oxygen concentration based on the measurement data of the acceleration sensor.

12. A measuring system according to claim 1,
wherein the calculation and control unit is configured to include in the determination of the oxygen concentration a measured value of a temperature sensor, which indicates a temperature level in the measuring chamber, and/or information provided with regard to a temperature level in the breathing gas mixture, and/or
wherein the calculation and control unit is configured to include in the determination of the oxygen concentration a measured value of a humidity sensor, which indicates a humidity situation in the breathing gas mixture in the measuring chamber, and/or information provided with regard to a current humidity level in the breathing gas mixture, taking into account in the determination of the oxygen concentration a measured value of a humidity sensor, which indicates a humidity situation in the breathing gas mixture in the measuring chamber, and/or information provided with regard to a current humidity content in the breathing gas mixture.

13. A measuring system according to claim 12,
wherein the temperature sensor is arranged in or on the measuring chamber, which indicates a temperature level in the measuring chamber, and/or
wherein the humidity sensor is arranged in or on the measuring chamber, which indicates a humidity level in the measuring chamber.

14. A measuring system according to claim 12,
wherein the calculation and control unit is configured in cooperation with the temperature sensor and with a measuring chamber heater to provide temperature control of the measuring chamber to a temperature level with a predetermined temperature difference above an ambient temperature or to a temperature level above the body temperature of the pilot for a predetermined period of time or to a selected temperature level for a specified period of time,
wherein the calculation and control unit is configured to select the temperature level, selected for the predetermined time period, based on a current temperature level in the measuring chamber and based on at least one of the data sets stored in the data memory.
